(19) United States
(12) Patent Application Publication
(10) Pub. No.: US 2026/0317545 A1
(43) Pub. Date: Oct. 15, 2026

(54) AROMATIC HYDROCARBON-SOLUBLE ANTHRAQUINONE

(71) Applicants: Dow Global Technologies LLC, Midland, MI (US); Rohm and Haas Company, Collegeville, PA (US)

(72) Inventors: Evelyn Auyeung, Houston, TX (US); Robert J. Wright, Sugar Land, TX (US); Brian A. Jazdzewski, Houston, TX (US); Nicole Knight, Rosharon, TX (US); Zahid Asif, Royersford, PA (US); Warren E. Smith, Buckinghamshire (GB)

(21) Appl. No.: 19/651,390

(22) Filed: Apr. 17, 2026

Related U.S. Application Data
(62) Division of application No. 18/004,707, filed on Jul. 19, 2021, now abandoned.
(60) Provisional application No. 63/057,928, filed on Jul. 29, 2020.

(51) Int. Cl. C09B 1/32; B01J 23/72; C10L 1/00

(52) U.S. Cl. CPC C09B 1/32; B01J 23/72; C10L 1/003; C10L 2200/0259

(57) ABSTRACT

The present invention is a method of making 1,4,5,8-tetrakis(R-phenylamino)anthracene-9,10-dione (where R is H or a hydrocarbyl group) comprising the step of contacting 1,4,5,8-tetrachloroanthraquinone with a mixture of R-anilines comprising at least two different R groups, under conditions to produce 1,4,5,8-tetrakis(R-phenylamino)anthracene-9,10-dione. The present disclosure also provides a reaction mixture of 1,4,5,8-tetrakis(R-phenylamino)anthracene-9,10-dione characterized by having the R groups on at least 75% of the 1,4,5,8-tetrakis(R-amino)anthracene-9,10-dione molecules in the mixture be comprised of 2 or more different R groups. The present disclosure also provides a method of improving the solubility of 1,4,5,8-tetrakis(R-phenylamino)anthracene-9,10-dione in aromatic hydrocarbons distilled from crude oil, comprising selecting a mixture of 1,4,5,8-tetrakis(R-phenylamino)anthracene-9,10-diones which are characterized by having at least 75% of the 1,4,5,8-tetrakis(R-phenylamino)anthracene-9,10-dione molecules in the mixture comprise two or more different R groups. The compounds of the present invention exhibit a suitable UV visible absorbance profile making them suitable for use as a fuel marker.

### BACKGROUND AND SUMMARY

The present disclosure provides a method of making 1,4,5,8-tetrakis(R-phenylamino)anthracene-9,10-dione (where R is H or a hydrocarbyl group) comprising the step of contacting 1,4,5,8-tetrachloroanthraquinone with a mixture of R-anilines comprising at least two different R groups, under conditions to produce 1,4,5,8-tetrakis(R-phenylamino)anthracene-9,10-dione. The present disclosure also provides a reaction mixture of 1,4,5,8-tetrakis(R-phenylamino)anthracene-9,10-dione characterized by having the R groups on at least 75% of the 1,4,5,8-tetrakis(R-amino)anthracene-9,10-dione molecules in the mixture be comprised of 2 or more different R groups. The present disclosure also provides a method of improving the solubility of 1,4,5,8-tetrakis(R-phenylamino)anthracene-9,10-dione in aromatic hydrocarbons distilled from crude oil, comprising selecting a mixture of 1,4,5,8-tetrakis(R-phenylamino)anthracene-9,10-diones which are characterized by having at least 75% of the 1,4,5,8-tetrakis(R-phenylamino)anthracene-9,10-dione molecules in the mixture comprise two or more different R groups. The anthraquinones of the present invention exhibit a suitable absorbance profile making them suitable for use as a fuel marker.

### BRIEF DESCRIPTION OF THE DRAWINGS

FIG. 1 is a chromatogram from LCMS of 1,4,5,8-tetrakis(R-amino)anthracene-9,10-dione prepared using a 50:50 molar mixture of p-toluidine and 4-n-butylaniline, purified by solvent washing.

### DEFINITIONS

The numerical ranges disclosed herein include all values from, and including, the lower value and the upper value. For ranges containing explicit values (e.g., “a range from 1 to 10”) any subrange between any two explicit values is included (e.g., the range 1-10 above includes subranges 1 to 2; 2 to 6; 5 to 7; 3 to 7; 5 to 6; etc.).

Unless stated to the contrary, implicit from the context, or customary in the art, all parts and percentages are based on weight, and all test methods are current as of the filing date of this disclosure.

The term “composition,” as used herein, refers to a mixture of materials which comprise the composition, as well as reaction products and decomposition products formed from the materials of the composition.

The terms “comprising,” “including,” “having,” and their derivatives, are not intended to exclude the presence of any additional component, step or procedure, whether or not the same is specifically disclosed. In order to avoid any doubt, all compositions claimed through use of the term “comprising” may include any additional additive, adjuvant, or compound, whether polymeric or otherwise, unless stated to the contrary. In contrast, the term, “consisting essentially of” excludes from the scope of any succeeding recitation any other component, step or procedure, excepting those that are not essential to operability. The term “consisting of” excludes any component, step or procedure not specifically delineated or listed.

The term “hydrocarbyl” means a moiety comprising carbon and hydrogen atoms.

### DETAILED DESCRIPTION

The present disclosure provides a 1,4,5,8-tetrakis(R-phenylamino)anthracene-9,10-dione characterized by having at least two different R groups The desired product therefore corresponds to the following formula, where each R is independently H or a C₁₋₂₀ hydrocarbyl, and where at least two different R groups are present.

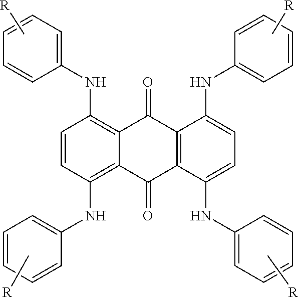

The R groups can independently be hydrogen or linear, branched, or cyclic hydrocarbons having from 1 to 20 Carbon atoms. Preferred R groups have from 0 to 12 carbon atoms. Linear hydrocarbyl groups are more preferred, with n-butyl, n-propyl, ethyl and methyl groups being most preferred. Preferably, the R group is in the para-position from the linking NH group, although one or more of the R groups can be in the ortho- or meta-positions as well.

While the molecules of the present invention have at least two different R groups, it is possible to have three or even four different R groups on the molecule.

The present disclosure also provides a method of making 1,4,5,8-tetrakis(R-phenylamino)anthracene-9,10-dione (where R is H or a C₁₋₂₀ hydrocarbyl group) comprising the step of contacting 1,4,5,8-tetrachloroanthraquinone with a molar excess of a mixture of two or more different anilines. The mixture of anilines can comprise unsubstituted aniline and/or one or more mono, di, or tri hydrocarbyl substituted anilines, where the hydrocarbyl substituted group can independently be any C1 through C20 linear, branched, or cyclic hydrocarbyl group. The mixture of anilines may comprise more than 2 different anilines. Preferably the mixture of anilines comprises at least 25 molar percent of a second aniline, more preferably the mixture of anilines comprises approximately equal molar amounts of each different aniline in the mixture.

Preferably, the R group on the substituted anilines is in the para-position (that is the 4 position) from the NH group, although one or more can be in the ortho- or meta-positions as well.

An excess of aniline with respect to the 1,4,5,8-tetrachloroanthraquinone should be used because a portion of aniline may be consumed in a side reaction with the base (as described below) to form an acetamide byproduct that can be removed by solvent washing. It is preferred that the mixture be added in an amount of at least 4, preferably 15, more preferably at least 20 molar equivalents with respect to the 1,4,5,8-tetrachloroanthraquinone.

The reaction can advantageously be carried out in an organic solvent such as alcohol, for example, isobutanol at a temperature above the boiling point of the alcohol (e.g., above 108° C., the boiling point of isobutanol) but below the boiling point of the anilines in the given aniline mixture. Basic conditions are preferred so it is preferred that a base be added to the reaction mixture. Preferred bases are Group I acetates such as potassium acetate. Sodium phosphate (dibasic) was also found to be a suitable base that formed the desired product, even in the absence of catalytic copper. The base should be added in an excess amount with respect to the reactive sites on 1,4,5,8-tetrachloroanthraquinone. For example, greater than 4 molar equivalents of the base should be used.

A catalyst can be added in a suitable amount to achieve desired product in a reasonable reaction time. Preferred catalysts include Copper (II) salts, such as copper sulfate.

It has been discovered that when formulated for use as fuel markers, the molecules exhibiting molecular symmetry (such as the tetra-substituted products present in 6.25 mol % abundance in the hypothetical mixture shown below) exhibit lower solubility in aromatic hydrocarbons distilled from crude oil, such as Aromatic 200 solvent, and therefore should be present in the lowest possible quantity in the mixture. Purification of the crude reaction mixture by successive solvent washes (for example, isobutanol, methanol, water) is preferred to remove at least a portion of undesired materials such as tri-substituted dechlorinated impurities, an acetamide impurity, and metal salts.

It will be readily understood by those skilled in the art that the above method of making the 1,4,5,8-tetrakis(R-phenylamino)anthracene-9,10-dione will result in a reaction mixture with different R groups and different molar equivalents of each R group incorporated on each individual molecule. Thus for example, when using a 50:50 molar mixture of 2 different anilines, having an R group of X and Y respectively, assuming equal reactivity, the resulting reaction mixture would be expected to have approximately 6.25 mol % of molecules having four X groups, 25 mol % of molecules having three X groups and one Y group, 37.5 mol % of molecules having two X groups and two Y groups, 25 mol % of molecules having three X groups and one Y group, and 6.25 mol % of molecules having four Y groups. The preferred reaction mixtures in the present invention can be characterized by having at least 75 mol %, more preferably 80 mol % or even 85 mol % of its molecules having at least 2 different R groups. Similarly, the preferred reaction mixtures in the present invention can be characterized by having no more than 25 mol %, more preferably 20 mol % or even 15 mol % of molecules having all four R groups be identical.

When the molecules or reaction mixture of the present invention are used as a fuel marker, they are typically added to a mixture of aromatic hydrocarbons distilled from crude oil, such as Aromatic 200. To ensure desired solubility in such solvent, the 1,4,5,8-tetrakis(R-phenylamino)anthracene-9,10-dione added should be selected such that at least 75 mol %, more preferably 80 mol % or even 85 mol % of its molecules having at least 2 different R groups.

The molecules of the present invention can optionally also be subjected to a cyanation or nitration reaction as generally known in the art (see, for example, U.S. Pat. No. 6,977,177) such that the composition of the present invention may be described by the following formula:

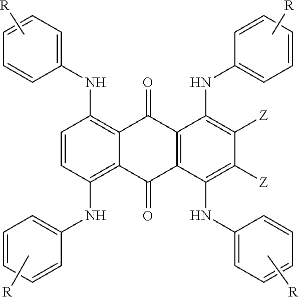

where each R is independently hydrogen or a linear, branched, or cyclic hydrocarbon having from 1 to 20 carbon atoms which may be located in either the para, ortho or meta position, and Z is CN or NO₂. The cyano and/or nitro derivatives will have a shifted UV-Vis spectra from the non-substituted anthraquinone derivates (Z═H), allowing for a differentiated fuel marker.

### Examples

A series of experiments are conducted to compare the efficacy of the present invention against a molecule derived from a single aniline, e.g. 1,4,5,8-tetrakis(4-butylphenylamino)anthracene-9,10-dione.

For each inventive example, a one liter round bottom flask is charged with the following: an egg-shaped magnetic stir bar, p-toluidine (that is, 4-methylaniline) (in the amounts indicated in table 1), 4-n-butylaniline (in the amounts indicated in table 1), 1,4,5,8-tetrachloroanthraquinone (12.00 g, 34.4 mmol), potassium acetate (13.54 g, 138 mmol), and copper sulfate (0.136 g, 0.86 mmol) along with isobutanol (160 mL). The mixture is heated at 130° C. for 2 hours, followed by heating at 170° C. for 4 hours, during which the isobutanol is removed by distillation. The comparative examples are similarly prepared except that in Example 4 no p-toluidine is used, and in Example 5, no 4-n-butylaniline is used.

TABLE 1

| | | | Total |
|---|---|---|---|
| | p-toluidine | 4-n-butylaniline | aniline |
| Example 1 | 18.5 g, 172.4 mmol | 75.6 g, 517.2 mmol | 689.6 mmol |
| Example 2 | 37 g, 344.8 mmol | 50.4 g, 344.8 mmol | 689.6 mmol |
| Example 3 | 55.5 g, 517.2 mmol | 25.2 g, 172.4 mmol | 689.6 mmol |
| Example 4 | 0 | 100.8 g, 689.6 mmol | 689.6 mmol |
| (compar- | | | |
| ative) | | | |
| Example 5 | 74 g, 689.6 mmol | 0 | 689.6 mmol |
| (Compar- | | | |
| ative) | | | |

The reaction mixture turns dark green and appears complete based on ¹H NMR, LCMS, and UV/vis, analysis which all show the presence of approximately 5% tri-substituted, dehalogenated product as an impurity. The acetamide byproducts of the reaction of aniline with base are also observed in both LCMS and GCMS. The mixture is cooled to ambient temperature and hexane (300 mL) and methanol (300 mL) are added. The mixture is stirred for 30 minutes and filtered. The filtrate is washed twice with water (750 mL). The solid is dried under reduced pressure to afford 13.1 g of product (53.4% yield). An alternative workup is as follows: The crude reaction mixture (before treatment with washing solvents) is subjected to reduced pressure to remove excess anilines (63 g). Methanol (200 mL) followed by hexanes (200 mL) is added and stirred at 60° C. for an unoptimized time of 3 days. Solvent (250 mL) is removed under reduced pressure. The precipitated crude product is filtered, washed twice by stirring with water (750 mL) for 30 min each, and is dried under reduced pressure. Residual aniline is removed by washing with 5% HCl (300 mL) twice, then water (300 mL), dried, washed once more with methanol (200 mL), and dried under reduced pressure to afford 21.2 g of product (89.2% yield).

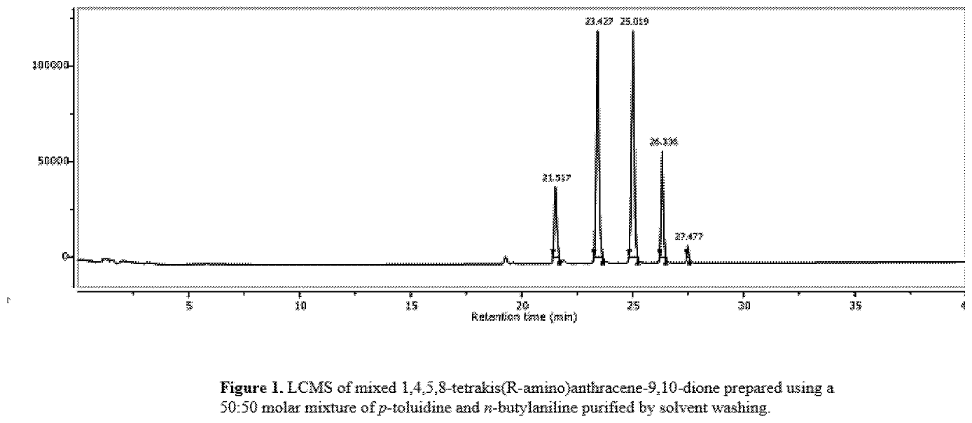

FIG. 1 shows a liquid chromatograph mass spectrometry (LCMS) chromatogram of Example 2 (the example prepared with a 50:50 molar blend of 4-methyl- and 4-n-butyl aniline) showing the expected 5 components, demonstrating that the method produced having a majority of 1,4,5,8-tetrakis(R-phenylamino)anthracene-9,10-dione molecules have at least 2 different R groups.

Storage stability experiments of Examples 1-3 as well as Comparative Examples 4 and 5 as well as Comparative Example 6 (a 50:50 molar physical blend of Comparative Examples 4 and 5) are conducted as follows. For each Example, a calibration line is generated by preparing 0.5 and 1.7 wt % solutions of each Example using a batch of Aromatic 200 supplied from TOTAL, identified as ATOSOL 200ND, and measuring the UV/vis absorbance at these known concentrations. The solutions are heated to 60° C. with stirring overnight and allowed to cool to ambient temperature for at least several hours, preferably overnight. For the 0.5 and 1.7 wt % solutions for each marker, UV/vis measurements are prepared by diluting an aliquot of each solution (exact weight recorded) into a known amount of xylenes to target an absorbance value below approximately 2.5. Examples that are not fully soluble at the concentrations needed for the calibration line are denoted in Table 2 as “Solids observed at 1.7 wt %, no measurement”. Higher concentration solutions (target=10 wt %) of each of the 1,4,5,8-tetrakis(R-phenylamino)anthracene-9,10-dione Examples in Aromatic 200 are prepared to determine the maximum concentration and storage stability of these materials. Note that solids are observed at 1.7 wt % for both Comparative Examples 5 and 6, therefore preparation of a 10 wt % solution is not attempted for those examples. For other Examples, several drops of the targeted 10 wt % solution are filtered and a known amount of the filtrate is diluted with a known amount of xylenes to target an absorbance value below approximately 2.5. The initial concentration is determined by measuring the absorbance value of a known concentration of the filtered and diluted aliquot taken from the targeted 10 wt % solution. The expected UV/vis absorbance value of the aliquot is calculated based on the calibration standards, and this value is compared against the actual measured absorbance value. Measured values below the expected value indicate the marker is not fully soluble at this 10 wt % concentration. The actual concentration of the soluble portion is calculated based on the calibration standards, and the values are shown in Table 2. The remaining solution is placed in the −10° C. freezer and storage stability measurements are taken after 4 days.

The results of this study is presented in Table 2.

TABLE 2
Storage stability data of MD-7 and examples synthesized using
different amounts of anilines in Aromatic 200 solvent

| | Measured | Storage stability: |
|---|---|---|
| | Initial | Concentration after |
| | Concentration, | storage at −10 |
| | wt % | °C, 4 days, wt % |
| Example 1: Mixed R Groups | 7.0 | 6.8 |
| (4-methylaniline/4-(n-butyl)aniline | | |
| 25:75) | | |
| Example 2: Mixed R Groups | 10 | 10 |
| (4-methylaniline/4-(n-butyl)aniline | | |
| 50:50) | | |
| Example 3: Mixed R groups | 7.5 | 6.2 |
| (4-methylaniline/4-(n-butyl)aniline | | |
| 75:25) | | |
| Comparative Example 4 (1,4,5,8- | 7.0 | 4.2 |
| tetrakis(4-(n-butyl)phenyl- | | |
| amino)anthracene-9,10-dione) | | |
| (incumbent) | | |
| Comparative Example 5: 1,4,5,8- | Solids observed | — |
| tetrakis(4-methylaniline)anthra- | at 1.7%, no | |
| cene-9,10-dione (4) | measurement | |
| Comparative Example 6 Physical | Solids observed | — |
| mixture Comparative Examples 4 | at 1.7%, no | |
| and 5) (50:50 molar ratio) | measurement | |

What is claimed is:

1. A method for marking a liquid petroleum hydrocarbon comprising the the step of adding a reaction mixture to a liquid petroleum hydrocarbon, wherein the reaction mixture results from contacting 1,4,5,8-tetrachloroanthraquinone with a mixture of two or more different R-anilines, under conditions to produce 1,4,5,8-tetrakis(R-phenylamino)anthracene-9,10-dione, where each R independently represents a linear, branched, or cyclic hydrocarbon having from 1 to 20 carbon atoms located in either the para, ortho or meta position, characterized by having the R groups on at least 75% of the 1,4,5,8-tetrakis(R-phenylamino)anthracene-9,10-dione molecules in the mixture be comprised of two or more different R groups.

2. The method of claim 1 where the reaction mixture is first added to an assortment of aromatic hydrocarbons distilled from crude oil to form a concentrated solution which is then added to the liquid petroleum hydrocarbon to be marked.

3. The method of claim 1 wherein less than 15% of the 1,4,5,8-tetrakis(R-phenylamino)anthracene-9,10-dione molecules in the mixture have all four R groups identical to each other.

4. The method of claim 1 wherein at least one of the anilines is a 4-alkyl aniline.

5. The method of claim 1 wherein the mixture of anilines comprises 4-n-butylaniline and 4-methylaniline.

6. The method of claim 1 wherein the mixture of anilines comprises 4-methylaniline in an amount of from 25% to 75% by molar percent of the mixture of anilines.

7. The method of claim 1 wherein the mixture of anilines comprises 4-n-butylaniline in an amount of from 25% to 75% by molar percent of the mixture of anilines.

8. The method of claim 1 wherein the different anilines in the mixture of anilines are present in approximately equal (within 10%) molar amounts.

9. The method of claim 1 wherein the mixture of anilines is added in amount of at least 4 equivalents with respect to the 1,4,5,8-tetrachloroanthraquinone.

10. The method of claim 1 wherein the mixture of anilines comprises from 2 to 4 different anilines.

11. The method of claim 1 wherein the 1,4,5,8-tetrachloroanthraquinone is contacted with a mixture of anilines in the presence of a base and optionally a catalyst.

12. The method of claim 11 wherein the base is a Group I acetate and the catalyst is present and is a copper (II) salt.

13. The method of claim 12 wherein the base is potassium acetate and the catalytic copper is copper sulfate.

14. The method of claim 11 wherein the base is added in an amount of at least 4 equivalents with respect to the 1,4,5,8-tetrachloroanthraquinone.

15. The method of claim 1 wherein the 1,4,5,8-tetrachloroanthraquinone is contacted with a mixture of anilines in an organic solvent which is above the boiling point of the organic solvent and below the boiling point of each aniline in the mixture of anilines.

16. The method of claim 15 wherein the the organic solvent is isobutanol.

17. The method of claim 1 further comprising a step of washing the reaction mixture with a solvent to remove at least some impurities and/or the 1,4,5,8-tetrakis(R-phenylamino)anthracene-9,10-dione groups which have all R groups being the same, prior to adding the reaction mixture to the liquid petroleum hydrocarbon.

18. The method of claim 2 further comprising a step of washing the reaction mixture with a solvent to remove at least some impurities and/or the 1,4,5,8-tetrakis(R-phenylamino)anthracene-9,10-dione groups which have all R groups being the same, prior to adding the reaction mixture to the assortment of aromatic hydrocarbons distilled from crude oil to form the concentrated solution.
